(19) United States
(12) Patent Application Publication
(10) Pub. No.: US 2008/0312608 A1
(43) Pub. Date: Dec. 18, 2008

(54) Cannula With Ductile Portion

(75) Inventors: Lasse Wengel Christoffersen, Soborg (DK); Henrik Sonderskov Klint, Lyngby (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: NOVO NORDISK A/S, BAGSVAERD (DK)

(21) Appl. No.: 11/917,048

(22) PCT Filed: Jun. 20, 2006
(86) PCT No.: PCT/EP2006/005895

§ 371 Date: Jul. 15, 2008

(30) Foreign Application Priority Data
Jun. 20, 2005 (EP) 05013245.5

(51) Int. Cl. A61M 5/32; A61M 5/34; B23K 26/00

(52) U.S. Cl. 604/239; 604/272; 604/240; 219/121.85

(57) ABSTRACT

The present invention relates to a new type of cannula for syringes or injection devices. The cannula according to the present invention comprises a substantially hard portion and a ductile portion axially arranged therewith, where the substantially hard portion comprises a tip which is adapted to penetrate the skin of a patient. The present invention also relates to an injection device comprising such cannula. The present invention further relates to a method for manufacturing a cannula for an injection device, the method comprising the steps of 1): providing a cannula of a hardened material, the cannula comprising a tip portion, and 2): exposing a part of the cannula other than the tip portion to heat in order to form a ductile portion.

The present invention relates to a cannula having a ductile portion. In particular, the present invention relates to a cannula having a substantially hard tip portion and a ductile portion axially arranged therewith.

### BACKGROUND OF THE INVENTION

Within the area of syringes and/or injection devices repeated bending of cannulas may cause cannulas to break—in worst case in the tissue of the patient using the syringe or injection device. The most vulnerable point of a cannula is near the point wherein the cannula is fixed.

Bending is assumed to occur especially when a patient by accident drops the syringe or injection device, picks the syringe or injection device up and straightens out a bended cannula. Bend cannulas should in principle never be straightened out, but a relatively large group of patients are however tempted to do so. Straightening a bended cannula typically induces severe strains in the cannula whereby microscopic cracks are formed in the surface of the cannulla. These cracks propagate rapidly when the cannula is exposed to additional strain and the risk of breaking a cannula and in worst leave it in the tissue of a patient is significantly increased.

Also, since the development of cannulas goes in the direction of cannulas with thinner walls, i.e. smaller tip diameter and larger inner diameter, microscopic tracks in the surface of the cannula become more critical.

It is not advantageous just to harden the cannulas for example by precipitation hardening or deformation hardening since a hardening near the fixation point of a cannula significantly increases the risk of fracture.

A solution to the above-mentioned problem would be to design a cannula with varying or different mechanical properties along the axial direction of the cannula. Such types of cannulas have been disclosed in connection with various applications in the patent literature. For example, U.S. Pat. No. 6,422,865 discloses an endodontic irrigator tip having a cannula with an annealed distal portion and a proximal portion which enables the cannula to easily move within a root canal of a tooth. The cannula extends from a hub that is adapted for coupling with a syringe or other delivery device. The cannula has an outlet orifice for delivering an irrigant out of the endodontic irrigator tip and into the root canal. The outlet orifice may be defined by a rounded rim that optimally enables the distal insertion end of the cannula to be advanced within the root canal.

The annealed distal portion of the cannula is flexible and bendable and may thus easily reach the root of a tooth via the root canal of the same tooth. The proximal, i.e. the non-annealed portion of the cannula, is kept relatively stiff in order to keep control of the distal portion of the cannula.

The cannula suggested in U.S. Pat. No. 6,422,865 is specially designed to reach the root of a tooth. Since the bendable portion of the cannula suggested in U.S. Pat. No. 6,422,865 is the outermost portion of the cannula this cannula will not solve the problem mentioned above, i.e. the problem relating to cracks formed in cannulas that have been straightened out after for example an accidental drop.

WO 2005/068000 relates to an injection needle for introducing a product into the human or animal body. The needle suggested in WO 2005/068000 comprises a distal needle section with a needle point and a proximal needle section. The distal and proximal needle sections are axially arranged along the injection needle in such a way that the proximal needle section must penetrate the skin in order to introduce the product. The injection needle according to WO 2005/068000 is characterised in that the distal needle section has greater flexural rigidity than the proximal needle section.

It is a general problem that conventional cannulas as suggested in U.S. Pat. No. 6,422,865 and WO 2005/068000 are not suitable for being straightened out in case they are accidentally bended. Thus, if a conventional cannula is straightened out, the risk of breaking the cannula the next time the cannula is inserted into the tissue of a patient is significantly increased due to fractures in the cannula. If a cannula breaks while being positioned in the tissue of the patient, the patient should in worst case undergo surgery in order to have the broken cannula removed.

It is an object of the present invention to provide a cannula for syringes or injection devices, the cannula being capable of being straightened out in case the cannula is accidentally bended, for example if the syringe or injection device is accidentally dropped.

It is a further object of the present invention to provide a cannula for syringes or injection devices, the cannula being capable of being straightened out an increased number of times without introducing factures in the cannula.

### SUMMARY OF THE INVENTION

The above-mentioned object is complied with by provided, in a first aspect, a cannula for an injection or a syringe device, the cannula comprising a substantially hard portion and a ductile portion axially arranged therewith, wherein the substantially hard portion comprises a tip which is adapted to penetrate the skin of a patient.

Thus, in its most general aspect the present invention relates to a cannula having two tube portions arranged axially with respect to each other. The outermost portion of the tube, which comprising the tip, is a relatively hard and non-bendable portion whereas the other portion of the tube, the ductile portion axially arranged therewith, is softer and thereby more flexible. The injection device to which the cannula is to be secured or fastened may be an injection device suitable for injecting set doses of a medicament, such as set doses of insulin.

In order to be able to secure or fasten the cannula according the present invention to a syringe or an injection device a connection element may be attached to the ductile portion of the cannula. Thus, the ductile portion of the cannula may be arranged between the substantially hard portion and the connection element. The connection element may be made of a polymer-based material, such as polypropylene. Alternatively, the injection device may be a syringe, such as a disposable syringe, where the cannula forms an integral, and thereby non-detachable, part of the disposable syringe.

The substantially hard portion of the cannula may have a hardness of at least 400 HV0.025, whereas the ductile portion of the cannula may have a hardness below 400 HV0.025, such as within the range 150-350 HV0.025.

The hardness of the substantially hard portion relative to the ductile portion may alternatively be characterised by the difference in hardness of the two sections. Thus, the difference in hardness between the substantially hard portion of the cannula and the ductile portion of the cannula may be at least 100 HV0.025. For example, the substantially hard portion may have a hardness of 600 HV0.25 whereas the ductile portion may have a hardness of 500H0.25.

In a cross-sectional profile of the ductile portion of the cannula, i.e. along a profile taken perpendicular to the axial direction of the cannula, the material forming for walls at the position of the ductile of the cannula may be homogeneous and uniform. Alternatively, the ductile portion may be formed by varying or changing the mechanical properties of the material forming the walls at the position of the ductile portion of the cannula along the axial direction of the cannula. Thus, it may be that for example the inner region of the cannula is made of a hardened material whereas the surrounding outer region is made of a softer material.

The cannula according to the present invention may have a free length in the range 3-12 mm, such as 4-12 mm, such as approximately 6 mm or approximately 8 mm. By free length is meant the length of the cannula extending from the connection element. The length of the ductile portion may be in the range 2-4 mm, such as approximately 3 mm. However, the ductile portion may be even shorter, such as down to 0.1 mm

In a second aspect, the present invention relates to a method for manufacturing a cannula for an injection or a syringe device, the method comprising the steps of
providing a cannula of a hardened material, the cannula comprising a tip portion, and
exposing a part of the cannula other than the tip portion to heat in order to form a ductile portion.

The exposure of heat may be applied to a portion of the cannula arranged between the tip portion and a connection element adapted to secure the cannula to the injection device. The latter may be made of a polymer-based material.

The heat applied to the cannula in order to form the ductile portion may be provided by a focussed laser beam from a laser, such as a CO₂ laser. Since the spot or beam waist of a focussed laser beam has a diameter significantly smaller than the area to be exposed to heat the laser beam needs to be scanned or moved relative to the cannula in order to cover the full area to be exposed and thereby heated. Alternative, the laser beam can be slightly defocused whereby the area to be exposed may be exposed in one shot.

Alternatively, the heat may be applied by locally exposing the cannula to an electron beam, or by exposing a part or parts of the canulla to an alternating magnetic field as known from induction hardening of carbon steels.

The heat locally applied to the canulla may be applied in such a way that the deformation hardening is fully or partially removed through the entire wall thickness at the position of the ductile portion of the cannula. In this way a homogeneous and uniform change of the wall of the canulla is reached.

Alternatively, the heat locally applied to the cannula may be applied in such a way that the deformation hardening is fully or partially removed through only part of the wall thickness at the position of the ductile portion of the cannula. According to this embodiment of the present invention only part of the wall at the position of the ductile of the cannula may be softened. This may in principle be any part of the wall, but typically the inner part is kept hardened whereas the outer part surrounding the hardened inner part is softened by the exposure of heat.

In a third aspect, the present invention relates to a method for manufacturing a cannula for an injection or a syringe device, the method comprising the steps of
providing a cannula of a ductile material, the cannula comprising a tip portion and a portion adapted for fixation to a connection element, and
hardening the tip portion of the cannula and leaving the fixation portion of the cannula as a ductile portion.

In a fourth aspect, the present invention relates to an injection device comprising a cannula according to the first aspect of the present invention, the injection device further comprising means for holding a medicament containing reservoir. The medicament in the reservoir may be insulin.

In a fifth aspect, the present invention relates to a cannula assembly comprising
a cannula having a substantially hard portion and a ductile portion axially arranged therewith, wherein the substantially hard portion comprises a tip which is adapted to penetrate the skin of a patient, and
a connection element adapted to secure the cannula to an associated injection device,
   wherein the ductile portion of the cannula is arranged between the substantially hard portion and the connection element.

The connection element may be made of a polymer based material, such as polypropylene. The substantially hard portion of the cannula may have a hardness of at least 400 HV0.025, whereas the ductile portion of the cannula may have a hardness below 400 HV0.025, such as within the range 150-350 HV0.025. The hardness of the substantially hard portion of the cannula may be at least 100 HV0.025 higher than the hardness of the ductile portion of the cannula.

The cannula may have a free length in the range 4-12 mm, such as approximately 6 mm or 8 mm, whereas the length of the ductile portion may be in the range 2-4 mm, such as approximately 3 mm. Alternatively, the length of the ductile portion may be in the range 0.1-0.5 mm, such as approximately 0.2 mm.

### BRIEF DESCRIPTION OF THE DRAWINGS

The present invention will now be explained in further details with reference to the accompanying figures, wherein

FIG. 1 shows a cannula having a tube and a polypropylene hub,

FIG. 2 shows a first embodiment of the present invention,

FIG. 3 shows a second embodiment of the present invention, and

FIG. 4 shows a third embodiment of the present invention.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION OF THE INVENTION

In its most general aspect the present invention relates to a cannula having a hardened outer portion and a ductile inner portion axially arranged therewith. The free length of the hardened portion and the ductile portion is between 4 and 12 mm whereas the length of the ductile portion alone is between 2 and 4 mm. The cannula according to the present invention is intended for use in connection with injection devices or syringes for injecting a medicament such as insulin. The present invention also relates to a cannula assembly comprising a cannula as mentioned above, and a connection element to which the cannula is fixedly arranged.

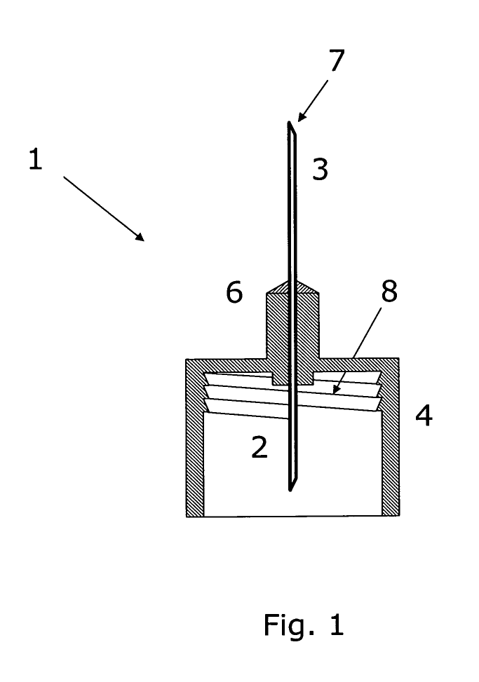

FIG. 1 shows a cannula 1 suitable for being mounted on an injection device (not shown) or syringe (also not shown). The cannula of FIG. 1 comprises an internal tube portion 2, an outer tube portion 3 and a polypropylene hub 4. The polypropylene hub 4 comprises a tube supporting section 6 to which the tube itself is secured using for example epoxy. The length of the outer tube portion 3 is typically 4-12 mm. At the end of the outer tube portion 3 a tip 7 is formed. In order to secure the cannula to an injection device or a syringe threads 8 are formed in an inner portion of the polypropylene hub 4. These threads are adapted to engage with corresponding threads provided on the injection device or syringe. The internal tube portion 2 is adapted to penetrate a membrane of a medicament containing cartridge positioned inside the injection device of syringe.

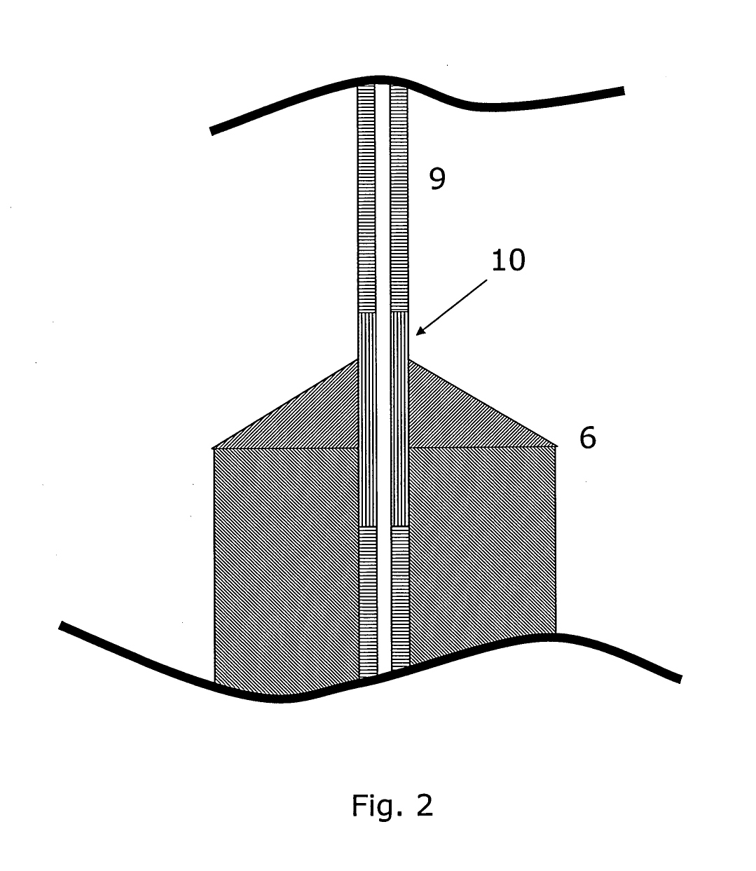

FIG. 2 shows a first embodiment of the present invention. FIG. 2 shows the tube supporting section 6 and tube portions 9 and 10. Tube portion 9 also includes the tip (not shown) of the tube. Tube portion 10 (the ductile portion) has a portion which is positioned inside the tube supporting section 6 and a portion which is outside the supporting section 6. The hardness of the tube portion 9 is at least 400 HV0.025, whereas the hardness of the tube portion 10 is below 400 HV0.025. Typically, the difference in hardness between portion 9 and portion 10 of the tube is at least 100 HV0.025. The difference in hardness' is obtained by applying heat to a selected portion or portions of the tube.

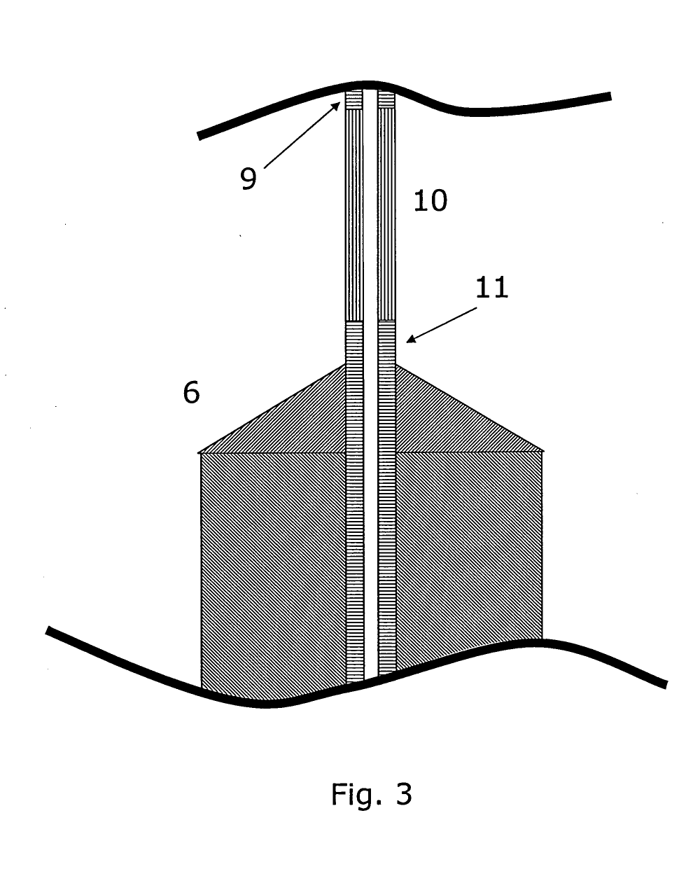

FIG. 3 shows a second embodiment of the present invention in that FIG. 3 shows the tube supporting section 6 and tube portions 9, 10 and 11. Tube portion 9 also includes the tip (not shown) of the tube. Tube portion 10 forms the ductile portion of the tube whereas tube portion 11 has a portion which is positioned inside the tube supporting section 6 and a portion which is outside the supporting section 6. The hardness of the tube portions 9 and 11 is at least 400 HV0.025, whereas the hardness of the tube portion 10 is below 400 HV0.025. Again, the difference in hardness between portions 9 and 11 and portion 10 is at least 100 HV0.025.

In both FIGS. 2 and 3 the ductile portion is formed by changing the mechanical properties of the tube through the entire wall thickness of the tube. Thus, the material forming the ductile portion of the tube is changed, i.e. softened by heating, throughout the entire wall thickness of the tube.

The ductile portion is typically provided into a hardened tube (having a hardness of at least 400 HV0.025) by applying heat to a selected portion of said tube. The heat applied to the tube in order to form the ductile portion can be provided by a focussed laser beam from a 100-1000 W CO₂ laser. However, other light sources such as diode lasers and YAG lasers are also applicable. The heating time would most likely be in the range 0.05 to 10 seconds.

Since the spot or beam waist of a focussed laser beam has a diameter significantly smaller than the area to be exposed to heat the laser beam needs to be scanned or moved relative to the cannula in order to cover the full area to be exposed and thereby heated. Alternative, the laser beam can be slightly defocused (for example to a spot diameter of approximately 3 mm) whereby the area to be exposed may be exposed in single-shot exposure. Alternatively, the heat can be applied by locally exposing the cannula to an electron beam, or by exposing a portion or portions of the canulla to an alternating magnetic field as known from induction hardening of carbon steels.

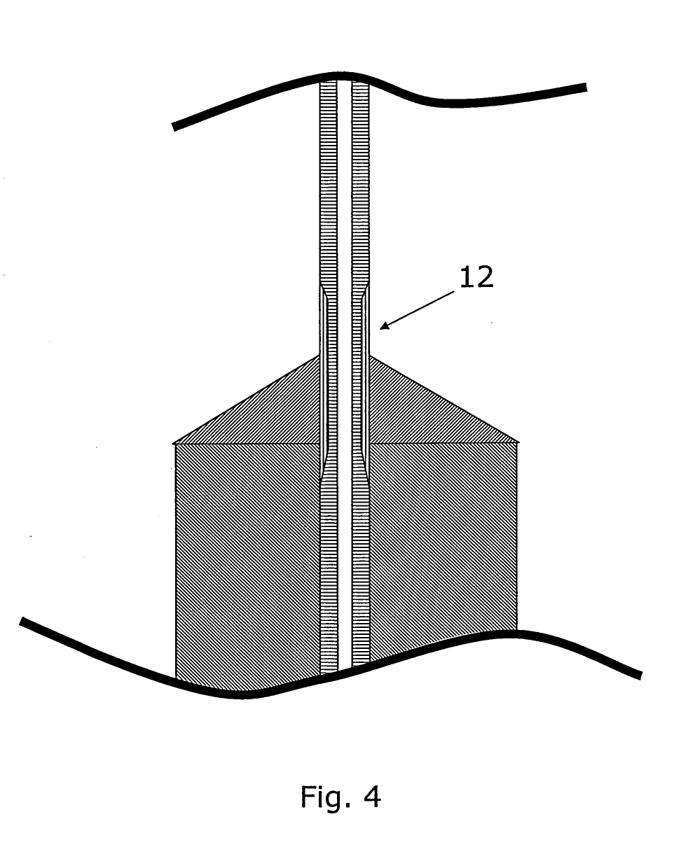

In FIG. 4 the heat locally applied to the cannula has been applied in such a way that the deformation hardening is fully or partially removed through only part of the wall thickness at the position of the ductile portion of the cannula. The ductile portion of the cannula in FIG. 4 is denoted 12. According to this third embodiment of the present invention only part of the wall at the position of the ductile of the cannula is softened. In FIG. 4 the softened region includes the outer surface of the cannula. However, this is just an example. Thus, the softened region may alternative include the inner surface of the cannula leaving a shell of hardened material around it.

To illustrate the effect of local heat treatment, deformation hardened stainless steel (AISI 304) cannulas having an inner and outer diameter of 0.13 mm and 0.26 mm, respectively, and a length of 16 mm were subjected to local heat treatment by passing the cannula at a predetermined velocity through a CO₂ laser beam. The size of the annealed zone is reported as the length of the heat affected zone (HAZ) measured along the cannula centre line. A range of HAZ lengths were obtained by changing the defocus, power and velocity during exposure. The HAZ centres were placed 6 mm from the tissue penetrating distal needle point, and the cannulas were glued into a connection element after heat treatment in such a way that 6 mm long free patient end needles were formed having a part of the HAZ placed in the glued portion of the cannulas.

The number of ±900 bends to fracture as well as the force causing yielding at 3 point loading were determined. During 3 point loading the cannula was supported by two pillars having a 5 mm span. The cannulas were positioned with the HAZ as midpoint between the pillars and were loaded at the HAZ by a plunger having a travel velocity of 1 mm/min. The used equipment was in compliance with ISO 9626:1991. Table 1 shows averages of 10 determinations on untreated and heat treated cannulas.

The chosen laser parameters increased the number of bends from 2.7 to 3.0-8.2. The yield load decreases from 2.6 N to 0.6-1.6 N, and the yield stress of the steel in the HAZ was therefore 23%-62% of the untreated deformation hardened steel.

TABLE 1

| | | | | Number of | |
|---|---|---|---|---|---|
| Parameter | Laser | Velocity | HAZ length | bends to | Bend yield |
| code | power (W) | (mm/min) | (mm) | fracture | load (N) |
| Untreated | — | — | — | 2.7 | 2.6 |
| L9 | 300 | 600 | 2.8 | 5.7 | 0.7 |
| L10 | 350 | 600 | 3.0 | 8.2 | 0.6 |
| L11 | 350 | 800 | 2.5 | 5.3 | 0.8 |
| L14 | 350 | 1200 | 1.5 | — | 1.2 |
| L16, 2 | 350 | 1200 | 3.0 | 4.0 | 1.3 |
| exposures | | | | | |
| L19 | 200 | 1000 | 1.0 | 3.0 | 0.9 |
| L34 | 350 | 1000 | 1.5 | — | 1.6 |

It is advantageous to implement the described heat treatment in conventional cannula grinding equipment. Cannula bevels can be formed by clamping 100-2000 tubes between to linear mechanical jaws thereby allowing a line of free tube ends to be grinded by a linear movement of a rotating grinding stone. The line of free tubing ends can be exposed to the laser beam before or after grinding. The time it takes to move the line of tubes through a laser beam can be optimised to match the time it takes to grind bevels, typically below 3 minutes. The local heat treatment can be incorporated in a grinding line thereby comprising a bevel grinding station, local heat treatment, burr removal and cleaning.

1. A cannula for an injection or a syringe device, the cannula comprising a substantially hard portion (9) and a ductile portion (10) axially arranged therewith, wherein the substantially hard portion (9) comprises a tip (7) which is adapted to penetrate the skin of a patient.

2. A cannula according to claim 1, further comprising a connection element (6) adapted to secure the cannula to the associated injection device.

3. A cannula according to claim 2, wherein the ductile portion (10) of the cannula is arranged between the substantially hard portion (9) and the connection element (6).

4. A cannula according to claim 2, wherein the connection element (6) is made of a polymer based material, such as polypropylene.

5. A cannula according to claim 1, wherein the substantially hard portion (9) of the cannula has a hardness of at least 400 HV0.025, and wherein the ductile portion (10) of the cannula has a hardness below 400 HV0.025, such as within the range 150-350 HV0.025.

6. A cannula according to claim 1, wherein the hardness of the substantially hard portion (9) of the cannula is at least 100 HV0.025 higher than the hardness of the ductile portion (10) of the cannula.

7. A cannula according to claim 1, wherein the cannula has al free length in the range 4-12 mm, such as approximately 6 mm or approximately 8 mm.

8. A cannula according to claim 1 wherein the length of the ductile portion (10) is in the range 2-4 mm, such as approximately 3 mm.

9. A cannula according to claim 1, wherein the length of the ductile portion (10) is in the range 0.1-0.5 mm, such as approximately 0.2 mm.

10. A method for manufacturing a cannula for an injection or a syringe device, the method comprising the steps of
providing a cannula of a hardened material, the cannula comprising a tip portion, and
exposing a part of the cannula other than the tip portion to heat in order to form a ductile portion.

11. A method according to claim 10, wherein the exposure of heat is applied to a portion of the cannula arranged between the tip portion and a connection element adapted to secure the cannula to the associated injection device.

12. A method according to claim 10, wherein a beam from a light source, such as a CO₂ laser, a YAG laser or a laser diode, is applied to a selected part or parts of the cannula in order to locally heat the cannula so as to form the ductile portion.

13. A method according to claim 10, wherein an electron beam is applied to a selected part or parts of the cannula in order to locally heat the cannula so as to form the ductile portion.

14. A method according to claim 10, wherein an alternating magnetic field is applied to a selected part or parts of the cannula in order to form the ductile portion.

15. A method according to claim 10, wherein the heat is applied in such a way that the deformation hardening is fully or partially removed through the entire wall thickness at the position of the ductile portion of the cannula.

16. A method according to claim 1, wherein the heat is applied in such a way that the deformation hardening is fully or partially removed through only part of the wall thickness at the position of the ductile portion of the cannula.

17. A method for manufacturing a cannula for an injection or a syringe device, the method comprising the steps of
providing a cannula of a ductile material, the cannula comprising a tip portion and a portion adapted for fixation to a connection element, and
hardening the tip portion of the cannula and leaving the fixation portion of the cannula as a ductile portion.

18. An injection device comprising a cannula according claim 1, the injection device further comprising means for holding a medicament containing reservoir.

19. A cannula assembly comprising
a cannula having a substantially hard portion (9) and a ductile portion (10) axially arranged therewith, wherein the substantially hard portion (9) comprises a tip (7) which is adapted to penetrate the skin of a patient, and
a connection element (6) adapted to secure the cannula to an associated injection device,
wherein the ductile portion (10) of the cannula is arranged between the substantially hard portion (9) and the connection element (6).

20. A cannula assembly according to claim 19, wherein the connection element (6) is made of a polymer based material, such as polypropylene.

21. A cannula assembly according to claim 19, wherein the substantially hard portion (9) of the cannula has a hardness of at least 400 HV0.025, and wherein the ductile portion (10) of the cannula has a hardness below 400 HV0.025, such as within the range 150-350 HV0.025.

22. A cannula assembly according to claim 19, wherein the hardness of the substantially hard portion (9) of the cannula is at least 100 HV0.025 higher than the hardness of the ductile portion (10) of the cannula.

23. A cannula assembly according to claim 19, wherein the cannula has a free length in the range 4-12 mm, such as approximately 6 mm or approximately 8 mm.

24. A cannula assembly according to claim 19, wherein the length of the ductile portion (10) is in the range 2-4 mm, such as approximately 3 mm.

25. A cannula assembly according to claim 19, wherein the length of the ductile portion (10) is in the range 0.1-0.5 mm, such as approximately 0.2 mm.
